(19) United States
(12) Patent Application Publication
(10) Pub. No.: US 2007/0010518 A1
(43) Pub. Date: Jan. 11, 2007

(54) Method for administering levosimendan

(76) Inventor: Matti Kivikko, Helsinki (FI)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/355,896

(22) Filed: Feb. 17, 2006

Related U.S. Application Data
(60) Provisional application No. 60/654,087, filed on Feb. 18, 2005.

(51) Int. Cl. A61K 31/50

(52) U.S. Cl. 514/247

(57) ABSTRACT

The invention relates to intermittent administration of a levosimendan compound or a pharmaceutically acceptable salt thereof in the treatment of cardiovascular disorders such as chronic heart failure. The intermittent dose is more than 0.04 mg/kg and the period between each intermittent dose is 6-30 days, preferably 6-25 days. The administration is suitably, for example, an intermittent intravenous infusion.

This application claims the benefit of priority to U.S. Provisional Application No. 60/654,087, filed on Feb. 18, 2005.

### FIELD OF THE INVENTION

The present invention relates to a method for the treatment of cardiovascular disorders such as chronic heart failure, pulmonary hypertension or myocardial ischemia by intermittent administration of a levosimendan compound, or a pharmaceutically acceptable salt thereof, to a patient.

### BACKGROUND OF THE INVENTION

Levosimendan, which is the (−)-enantiomer of [[4-(1,4,5,6-tetrahydro-4-methyl-6-oxo-3-pyridazinyl)phenyl]hydrazono]propanedinitrile, is an inotropic drug substance that is currently used for the short term treatment of patients who suffer from acutely decompensated severe heart failure. The drug increases contractile force of the heart myocardium by enhancing the sensitivity of myofilaments to calcium. Levosimendan is administered by intravenous infusion over the period of 24 hours.

Levosimendan and a method for its preparation are described in U.S. Pat. No. 5,569,657. Use of levosimendan in the treatment of myocardial ischemia is described in U.S. Pat. No. 5,512,572. Use of levosimendan in the treatment of pulmonary hypertension is described in U.S. Pat. No. 6,462,045.

Chronic heart failure is typically treated with various drugs including diuretics, digitalis, beta-blockers, ACE inhibitors, angiotensin II blockers and aldosterone inhibitors. However, patients with acutely decompensated severe heart failure require immediate parenteral inotropic support to reverse the severe loss of myocardial function and contractility. Such continuous intravenous inotropic support is usually a short-term treatment necessary to stabilize the patients and bring them out of a period of decompensation. Prolonged continuous infusion may be necessary in some patients including those waiting for cardiac transplantation. Prolonged continuous inotropic infusions may, however, be associated with drawbacks such as tolerance, weaning problems, increased risk of infections, long-term hospitalization and increased mortality.

### SUMMARY OF THE INVENTION

The present invention provides a method of administering a levosimendan compound or a pharmaceutically acceptable salt thereof to a patient, the method comprising administering intermittently a dose of more than 0.04 mg/kg of the levosimendan compound or a pharmaceutically acceptable salt thereof to a patient, wherein the period between each intermittent dose is from 6 to 30 days, preferably from 6to 25 days.

The present invention also provides a method of administering a levosimendan compound or a pharmaceutically acceptable salt thereof to a patient, the method comprising administering a first dose of levosimendan or a pharmaceutically acceptable salt thereof to a patient, followed by a rest period during which the levosimendan compound or a pharmaceutically acceptable salt thereof is not administered, and then administering a second dose of the levosimendan compound or a pharmaceutically acceptable salt thereof, wherein said rest period is from 6 to 30 days, and each dose delivering more than 0.04 mg/kg of the levosimendan compound or a pharmaceutically acceptable salt thereof to a patient.

The present invention also provides a method of administering a levosimendan compound or a pharmaceutically acceptable salt thereof to a patient, the method comprising administering to a patient intermittent intravenous infusions each infusion delivering a dose of more than 0.04 mg/kg of the levosimendan compound or a pharmaceutically acceptable salt thereof to a patient, and wherein the period between each intermittent infusion is from 6 to 30 days, preferably from 6 to 25 days.

The present invention also provides a kit that comprises:

a) a composition comprising a therapeutically effective amount of a levosimendan compound or a pharmaceutically acceptable salt thereof,

b) a package for containing said composition, and

c) instructions for administering intermittently doses of more than 0.04 mg/kg of the levosimendan compound or a pharmaceutically acceptable salt thereof to a patient, wherein the period between each intermittent dose is from 6 to 30 days, preferably from 6 to 25 days.

The method of the present invention provides a safe and effective short-term or long-term treatment of cardiovascular disorders, including but not limited to severe or less severe chronic heart failure. The method can be used also in the treatment of other cardiovascular diseases such as myocardial ischemia or pulmonary hypertension.

### DETAILED DESCRIPTION OF THE INVENTION

The method of the invention relates to a method of administering a levosimendan compound or a pharmaceutically acceptable salt thereof intermittently, i.e. by administering a plurality (i.e., two or more) of intermittent doses, to a patient in need thereof. The intermittent administration according to the present invention is effective in reversing hemodynamic and neurohormonal disturbances in patients suffering from cardiovascular diseases, particularly chronic heart failure.

In particular, the intermittent administration according to the present invention provide important long-term benefits, such as increased cardiac output and decreased left ventricular filling pressure, with minimal risks in chronic heart failure patients. Levels of natriuretic peptides such as ANP (atrial natriuretic peptide) and BNP (brain natriuretic peptide) or their fragments (NT-proATP, NT-proBNP) which reflect myocardial diastolic pressure load, are also effectively suppressed. It was found that the plasma levels of these peptides were particularly suitable in monitoring the intermittent treatment of patients with chronic heart failure and in selecting the most suitable rest period between the intermittent doses.

The method of the invention also provides reduced risk of drug tolerance and improves patient compliance. In the intravenous setting, the method of the invention reduces the drawbacks associated with prolonged or continuous infusions such as weaning problems, increased risk of infection, long-term hospitalization and increased mortality.

The term “intermittent” means administration that occurs non-continuously or at intervals. Intermittent administration encompasses dosing of an established amount of the drug, in a consecutive or non-consecutive manner, with a rest period of days or weeks, within the total administration period. For example, intermittent administration may occur once a week, once every second week, once every third week, and so on. The term intermittent also includes drug administration on consecutive days followed by period of days when no drug is administered. For example, the drug can be administered on day one and two and then again on day nine and ten, and so on. In a similar manner, the drug can be administered on day one and three (i.e. non-consecutively) followed by a period of days when no drug is administered. Thus, the intermittent dose can be administered over a period which ranges from minutes to several days. For example, if an intermittent dose is 0.3 mg/kg, it can be dosed consecutively over three days at a daily dose of 0.1 mg/kg. Alternatively, an intermittent dose of 0.3 mg/kg can be administered over three days as a first dose of 0.15 mg/kg, a second dose of 0.10 mg/kg and a third dose of 0.05 mg/kg. Many variations of intermittent administration will be apparent to those skilled in the art.

As used herein, the term “levosimendan compound” refers to any racemic mixture or enantiomer of levosimendan or a racemic mixture or enantiomer of the active metabolite of levosimendan. The term “levosimendan” specifically refers to the (−)-enantiomer of [4-(1,4,5,6-tetrahydro-4-methyl-6-oxo-3-pyridazinyl)phenyl]hydrazono]propanedinitrile. The term also is intended to encompass combinations of levosimendan and its active metabolite. The active metabolite of levosimendan is particularly (R)-N-[4-(1,4,5,6-tetrahydro-4-methyl-6-oxo-3-pyridazinyl)phenyl]acetamide.

The term “mg/kg of a levosimendan compound or a pharmaceutically acceptable salt thereof” means milligram of a levosimendan compound or a pharmaceutically acceptable salt thereof per one kilogram bodyweight of the patient, unless otherwise indicated. Similarly, the term “μg/kg of a levosimendan compound or a pharmaceutically acceptable salt thereof” means microgram of a levosimendan compound or a pharmaceutically acceptable salt thereof per one kilogram bodyweight of the patient.

The term “patient” means animals, preferably mammals, and humans.

The terms “treating”, “treat” or “treatment” includes preventive (e.g. prophylactic) and palliative treatment.

As used herein, the phrase “pharmaceutically acceptable” refers to a form of an ingredient that is physiologically suitable for pharmaceutical use. For example, the phrase “pharmaceutically acceptable salt” refers to the salt forms of an active ingredient, such as levosimendan, that is physiologically suitable for pharmaceutical use.

The strength of each intermittent dose to be administered to a patient depends e.g. upon the condition to be treated, the method of administration, age and the condition of the patient. In general, each intermittent dose comprises more than 0.04 mg/kg of a levosimendan compound or a pharmaceutically acceptable salt thereof. More preferably, each intermittent dose comprises from about 0.05 to about 1 mg/kg, for example from about 0.1 to about 0.6 mg/kg, of a levosimendan compound or a pharmaceutically acceptable salt thereof, depending on the period over which the intermittent dose is administered.

Typically, each intermittent dose is administered over a period which ranges from minutes to several days. Suitably, each intermittent dose is administered over the period, which is less than 7 days, preferably less than 5 days, and more preferably less than 3 days.

The administration of the intermittent dose can be by e.g. parenteral, oral, transmucosal or transdermal route.

In general, a levosimendan compound can be administered orally to man in a daily dose ranging from about 0.01 to about 0.2 mg/kg, more typically from 0.02 to 0.15 mg/kg, depending on the age, body weight and condition of the patient.

According to one preferred embodiment of the invention the administration of the intermittent dose is by intravenous route, suitably by intravenous infusion.

According to one particularly preferred embodiment of the invention the intermittent dose is given by an intravenous infusion over the period of 1-48 hours, preferably over the period of 4-36 hours, more preferably over the period of 6-30 hours, for example by an intravenous infusion lasting 24 hours, 12 hours, 8 hours or 6 hours. In another preferred embodiment of the invention, the intermittent dose is given by an intravenous infusion of less than 24 hours.

Suitably, the intermittent intravenous infusion is administered at the rate of 0.01-3 μg/kg/min, more preferably at the rate of 0.02-1 μg/kg/min, still more preferably at the rate of 0.03-0.4 μg/kg/min, of a levosimendan compound or a pharmaceutically acceptable salt thereof. If an initial intravenous bolus is needed, an intravenous bolus of 1-100 μg/kg, preferably 5-50 μg/kg, of a levosimendan compound or a pharmaceutically acceptable salt thereof, followed by maintenance infusion at the rate as described above can be used.

The period between each intermittent dose, i.e. the rest period during which drug is not administered, is from about 6 to about 30 days. Preferably, the period between each intermittent dose is from 6 to 25 days, more preferably from 7 to 22 days, still more preferably from about 7 to about 16 days, for example from 8 to 13 days.

Intermittent continuous infusion at the rate of 0.1-0.2 μg/kg/min, optionally with a initial loading dose of 10-15 μg/kg, of levosimendan or a pharmaceutically acceptable salt thereof for 24 hours followed by a rest period of 7 to 22 days, preferably from 7 to 16 days, more preferably from 8 to 13 days, has been found to be particularly suitable for the treatment of chronic heart failure, particularly severe chronic heart failure.

If desired, the length of the rest period, i.e. the period when no drug is administered, can be selected for each patient using suitable invasive or non-invasive monitoring means, non-invasive means being preferred. Such invasive and non-invasive means are well known to those of ordinary skill in the art. A particularly suitable non-invasive means is echocardiography. Furthermore, natriuretic peptides ANP (atrial natriuretic peptide), BNP (brain natriuretic peptide) and/or or their fragments (NT-proATP, NT-proBNP) are particularly suitable as surrogate markers and may be used to select the length of the rest period between intermittent infusions.

A levosimendan compound or a pharmaceutically acceptable salt thereof is formulated into dosage forms using principles well known to practitioners in the art. It is given to a patient as such or preferably in combination with suitable pharmaceutical excipients in the form of tablets, granules, capsules, suppositories, emulsions, suspensions or solutions whereby the contents of the active compound in the formulation is from about 0.1 to 100% per weight. Choosing suitable ingredients for the composition is routine for those of ordinary skill in the art. It is evident that suitable carriers, solvents, gel forming ingredients, dispersion forming ingredients, antioxidants, colours, sweeteners, wetting compounds, release controlling components and other ingredients normally used in this field of technology also may be used.

For oral administration in tablet or capsule form, suitable carriers and excipients include e.g. lactose, corn starch, magnesium stearate, calcium phosphate and talc. For controlled release oral compositions release controlling components can be used. Typical release controlling components include hydrophilic gel forming polymers such as hydroxypropylmethyl cellulose, hydroxypropyl cellulose, carboxymethyl celluloses, alginic acid or a mixture thereof; vegetable fats and oils including vegetable solid oils such as hydrogenated soybean oil, hardened castor oil or castor seed oil.

Tablets can be prepared by mixing the active ingredient with the carriers and excipients and compressing the powdery mixture into tablets. Capsules can be prepared by mixing the active ingredient with the carriers and excipients and placing the powdery mixture in capsules, e.g. hard gelatin capsules. Typically a tablet or a capsule comprises from about 0.1 to 10 mg, more typically 0.2 to 5 mg, of levosimendan or a pharmaceutically acceptable salt thereof.

Formulations suitable for intravenous administration such as injection or infusion formulation comprise sterile isotonic solutions of a levosimendan compound or a pharmaceutically acceptable salt thereof and vehicle, preferably a pharmaceutically acceptable aqueous solutions. Typically an intravenous infusion solution comprises from about 0.001 to 1, preferably from about 0.01 to 0.1 mg/ml, of a levosimendan compound or a pharmaceutically acceptable salt thereof. The formulation for intravenous administration may also be in the form of an infusion concentrate, which is diluted with an aqueous vehicle before use. Typically such infusion concentrate comprises a levosimendan compound or a pharmaceutically acceptable salt thereof dissolved in dehydrated ethanol. A preferred formulation is described in WO 01/19334, published Mar. 22, 2001.

The present invention also provides a kit comprising

a) a composition comprising a therapeutically effective amount of a levosimendan compound or a pharmaceutically acceptable salt thereof,

b) a package for containing said composition, and

c) instructions for administering intermittently doses of more than 0.04 mg/kg of the levosimendan compound or a pharmaceutically acceptable salt thereof to a patient, wherein the period between each intermittent dose is from 6 to 30 days, preferably from 6 to 25 days.

The composition of the above kit comprising a therapeutically effective amount of a levosimendan compound or a pharmaceutically acceptable salt thereof may be any of the formulations described above, e.g. in the form of tablets, granules, capsules, suppositories, emulsions, suspensions or solutions whereby the contents of the active compound in the formulation is from about 0.1 to 100% per weight. The package may be in any form normally used in the art, e.g. a bottle, blister, syringe, bag, box, and the like, depending on the nature of the composition. Typically, the kit comprises instructions for the intermittent administration of the composition in accordance to the method of the present invention.

Salts of levosimendan may be prepared by known methods. Pharmaceutically acceptable salts are useful as active medicaments, however, preferred salts are the salts with alkali or alkaline earth metals.

### EXAMPLES

### Pharmaceutical Examples

### EXAMPLE 1

### Oral Capsule:

### Hard Gelatin Capsule Size 3

| | |
|---|---|
| Levosimendan | 2.0 mg |
| Lactose | 198 mg |

The pharmaceutical preparation in the form of a capsule was prepared by mixing levosimendan with lactose and placing the powdery mixture in hard gelatin capsule.

### EXAMPLE 2

### Concentrate Solution for Intravenous Infusion

| | | |
|---|---|---|
| (a) levosimendan | 2.5 | mg/ml |
| (b) Kollidon PF12 | 10 | mg/ml |
| (c) citric acid | 2 | mg/ml |
| (d) dehydrated ethanol | ad 1 | ml (785 mg) |

The concentrate solution was prepared by dissolving citric acid, Kollidon PF121 and levosimendan to dehydrated ethanol in the sterilized preparation vessel under stirring. The resulting bulk solution was filtered through a sterile filter (0.22 μm). The sterile filtered bulk solution was then aseptically filled into 8 ml and 10 ml injection vials (with 5 ml and 10 ml filling volumes) and closed with rubber closures.

The concentrate solution for intravenous infusion is diluted with an aqueous vehicle before use. Typically the concentrate solution is diluted with aqueous isotonic vehicles, such as 5% glucose solution or 0.9% NaCl solution so as to obtain an aqueous intravenous solution, wherein the amount of levosimendan is generally within the range of about 0.001-1.0 mg/ml, preferably about 0.01-0.1 mg/ml.

### EXPERIMENTS

Methods

A double-blind, placebo-controlled, parallel group, single center study in patients with New York Heart Association functional class II to III heart failure was conducted. Patients were randomised in 1:1 ratio to receive either levosimendan or placebo. Study drug administration was initiated with a loading dose of 12 μg/kg of levosimendan or placebo delivered over 10 minutes. This was followed by a continuous infusion of 0.1 μg/kg/min for 50 minutes. If the dose was well tolerated the infusion rate was increased to 0.2 μg/kg/min for a further 23 hours. After the study drug was started, the hemodynamic assessments were repeated at 30 minutes, at two and six hours. The non-invasive hemodynamic assessments were repeated at 24-hours. Follow-up visits took place at 2, 3, 5, 7, 9 and 14 days from the beginning of the study. On these visits echocardiographic measurements were performed and blood pressure and heart rate were measured.

An Acuson Sequoia ultrasound system with 2.5-3.75 MHz probes was used for the Doppler echocardiographic measurements. Blood flow velocity curves were recorded at a sweep speed of 100 mm/s. Left ventricular ejection fraction was assessed by two-dimensional apical two- and four-chamber views with use of modified Simpson rule. Mitral flow velocity was assessed by pulsed wave Doppler from the apical four-chamber view by placing a 3-mm sample volume between the tip of the mitral leaflets in diastole. The following measurements were made over five consecutive cycles: maximal early and maximal late diastolic velocity and their ratio, duration of the late diastolic velocity wave, deceleration time and deceleration rate of early diastolic velocity. Peak velocities were defined as the highest point of the spectrum.

Blood samples (4 ml) for natriuretic peptide (NT-proANP, NT-proBNP) measurements were taken into pre-cooled EDTA tubes. Samples were taken at baseline (0-sample) and on the first study day 30 min, 2 h, 6h and 24 h after the start of the infusion and in the mornings of days 2, 3, 5, 7, 9 and 14 after the start of the study drug infusion.

Results

Invasively measured cardiac output (CO) increased from 4.3 L/min to 5.4 L/min in levosimendan group at six hours. PCWP decreased from 20 mmHg to 15 mmHg in response to the levosimendan treatment, whereas a small increase in PCWP (17 mmHg-20 mmHg) was observed in placebo group.

Echocardiographically estimated PCWP reached its lowest value 2 days after starting the infusion, whereas the highest CO value estimated by echocardiography was detected at the end of the 24-hour infusion. The linearly estimated duration of the decrease in PCWP was 9 days, and the duration of an increase in CO was 13 days. Plasma NT-proANP and NT-proBNP levels reached their lowest values at days 3 and 2, and by a linear model the treatment effect was estimated to last 16 and 12 days, respectively. NT-proANP and NT-proBNP levels closely coincided with the sustained hemodynamic response in the patients.

Conclusions: A 24-hour levosimendan infusion achieved a rapid improvement in the hemodynamic parameters of patients with NYHA II-III congestive heart failure with maximal effects occurring during 1-3 days after starting the infusion. The beneficial hemodynamic and neurohormonal effects were maintained up to two weeks after levosimendan administration. It is concluded that a 24-hour levosimendan infusion of 0.2 μg/kg/min given intermittently every 10 days is effective in achieving clinical benefits with minimal risks in patients suffering from severe chronic heart failure.

1. A method of administering levosimendan or a pharmaceutically acceptable salt thereof to a patient, the method comprising administering intermittently a dose of more than 0.04 mg/kg of levosimendan or a pharmaceutically acceptable salt thereof to a patient, wherein the period between each intermittent dose is from 6 to 25 days.

2. (canceled)

3. A method of administering levosimendan or a pharmaceutically acceptable salt thereof to a patient, the method comprising administering a first dose of levosimendan or a pharmaceutically acceptable salt thereof to a patient, followed by a rest period during which levosimendan or a pharmaceutically acceptable salt thereof is not administered, and then administering a second dose of levosimendan or a pharmaceutically acceptable salt thereof to the patient, wherein said rest period is from 6 to 25 days, and wherein each dose delivers more than 0.04 mg/kg of levosimendan or a pharmaceutically acceptable salt thereof to the patient.

4. (canceled)

5. A method of administering levosimendan or a pharmaceutically acceptable salt thereof to a patient, the method comprising administering to a patient intermittent intravenous infusions each infusion delivering a dose of more than 0.04 mg/kg of levosimendan or a pharmaceutically acceptable salt thereof to the patient, and wherein the period between each intermittent infusion is from 6 to 25 days.

6. (canceled)

7. A method according to claim 5, wherein the dose of each intermittent intravenous infusion is from 0.05 to 1 mg/kg of levosimendan or a pharmaceutically acceptable salt thereof.

8. A method according to claim 7, wherein the dose of each intermittent intravenous infusion is 0.1 to 0.6 mg/kg of levosimendan or a pharmaceutically acceptable salt thereof.

9. A method according to claim 5, wherein the administration of each intermittent intravenous infusion is over the period of 1-48 hours.

10. A method according to claim 9, wherein the administration of each intermittent intravenous infusion is over the period of 4-36 hours.

11. A method according to claim 10, wherein the administration of each intermittent intravenous infusion is over the period of 6-30 hours.

12. A method according to claim 6, wherein the period between each intermittent dose is from 7 to 22 days.

13. A method according to claim 12, wherein the period between each intermittent dose is from 7 to 16 days.

14. A method according to claim 13, wherein the period between each intermittent dose is from 8 to 13 days.

15. A method according to claim 5, wherein each intermittent intravenous infusion is administered at the rate of 0.01-3 μg/kg/min of levosimendan or a pharmaceutically acceptable salt thereof.

16. A method according to claim 15, wherein each intermittent intravenous infusion is administered at the rate of 0.02-1 μg/kg/min of levosimendan or a pharmaceutically acceptable salt thereof.

17. A method according to claim 16, wherein each intermittent intravenous infusion is administered at the rate of 0.03-0.4 μg/kg/min of levosimendan or a pharmaceutically acceptable salt thereof.

18. A method according to claim 1, which comprises performing an echocardiography on the patient to select the period between each intermittent dose.

19. A method according to claim 1, which comprises measuring the patient's plasma level of a natriuretic peptide or a fragment thereof to select the period between each intermittent dose.

20. A method according to claim 3, which comprises performing an echocardiography on the patient to select the period between each intermittent dose.

21. A method according to claim 3, which comprises measuring the patient's plasma level of a natriuretic peptide or a fragment thereof to select the period between each intermittent dose.

22. A method according to claim 5, which comprises performing an echocardiography on the patient to select the period between each intermittent dose.

23. A method according to claim 5, which comprises measuring the patient's plasma level of a natriuretic peptide or a fragment thereof to select the period between each intermittent dose.

24. A kit that comprises:
a) a composition comprising a therapeutically effective amount of levosimendan or a pharmaceutically acceptable salt thereof,
b) a package for containing said composition, and
c) instructions for administering intermittently doses of more than 0.04 mg/kg of levosimendan or a pharmaceutically acceptable salt thereof to a patient, wherein the period between each intermittent dose is from 6 to 25 days.

25. (canceled)

26. A method according to claim 5, wherein the administration of each intermittent intravenous infusion is over a period of less than 24 hours.

27. A method according to claim 1, which comprises administering at least one dose of levosimendan or a pharmaceutically acceptable salt thereof orally.

28. A method according to claim 3, which comprises administering at least one dose of levosimendan or a pharmaceutically acceptable salt thereof orally.
